SEQUENCE LISTING (43 sequences)

SEQ ID NO: 1 - protein, 719 aa, Peptostreptococcus magnus
MAALAGAIVVTGGVGSYAADEPIDLEKLEEKRDKENVGNLPKFDNEVKDGSENPMAKYPDFDDEASTRFETENNEFEEKKVVSDNFFDQSEHPFVENKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEKEQVTIKENIYFEDGTVQTATFKGTFAEATAEAYRYADLLSKEHGKYTADLEDGGYTINIRFAGKEEPEETPEKPEVQDGYASYEEAEAAAKEALKNDDVNKSYTIRQGADGRYYYVLSPVEAEEEKPEAQNGYATYEEAEAAAKKALENDPINKSYSIRQGADGRYYYVLSPVEAETPEKPVEPSEPSTPDVPSNPSNPSTPDVPSTPDVPSNPSTPEVPSNPSTPGNEEKPGNEQKPGNEQKPGNEQKPGNEQKPGNEQKPDQPSKPEKEENGKGGVDSPKKKEKAALPKAGSEAEILTLAAASLSSVAGAFISLKKRK

SEQ ID NO: 2 - protein, 992 aa, Peptostreptococcus magnus
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYRYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK

SEQ ID NO: 3 - protein, 76 aa, Peptostreptococcus magnus
KEETPETPETDSEEEVTIKANLIFANGSTQTAEFKGTFEKATSEAYAYADTLKKDNGEYTVDVADKGYTLNIKFAG

SEQ ID NO: 4 - protein, 72 aa, Peptostreptococcus magnus
KEKTPEEPKEEVTIKANLIYADGKTQTAEFKGTFEEATAEAYRYADALKKDNGEYTVDVADKGYTLNIKFAG

SEQ ID NO: 5 - protein, 72 aa, Peptostreptococcus magnus
KEKTPEEPKEEVTIKANLIYADGKTQTAEFKGTFEEATAEAYRYADLLAKENGKYTVDVADKGYTLNIKFAG

SEQ ID NO: 6 - protein, 72 aa, Peptostreptococcus magnus
KEKTPEEPKEEVTIKANLIYADGKTQTAEFKGTFAEATAEAYRYADLLAKENGKYTADLEDGGYTINIRFAG

SEQ ID NO: 7 - protein, 73 aa, Peptostreptococcus magnus
KKVDEKPEEKEQVTIKENIYFEDGTVQTATFKGTFAEATAEAYRYADLLSKEHGKYTADLEDGGYTINIRFAG

SEQ ID NO: 8 - protein, 71 aa, Peptostreptococcus magnus
KETPEPEEEVTIKANLIFADGSTQNAEFKGTFAKAVSDAYAYADALKKDNGEYTVDVADKGLTLNIKFAGK

SEQ ID NO: 9 - protein, 71 aa, Peptostreptococcus magnus
KEKPEEPKEEVTIKVNLIFADGKTQTAEFKGTFEEATAKAYAYADLLAKENGEYTADLEDGGNTINIKFAG

SEQ ID NO: 10 - protein, 74 aa, Peptostreptococcus magnus
KETPETPEEPKEEVTIKVNLIFADGKIQTAEFKGTFEEATAKAYAYANLLAKENGEYTADLEDGGNTINIKFAG

SEQ ID NO: 11 - protein, 75 aa, Peptostreptococcus magnus
KETPETPEEPKEEVTIKVNLIFADGKTQTAEFKGTFEEATAEAYRYADLLAKVNGEYTADLEDGGYTINIKFAGK

SEQ ID NO: 12 - protein, 60 aa, Peptostreptococcus magnus
VTIKANLIFANGSTQTAEFKGTFEKATSEAYAYADTLKKDNGEYTVDVADKGYTLNIKFA

SEQ ID NO: 13 - protein, 60 aa, Peptostreptococcus magnus
VTIKANLIYADGKTQTAEFKGTFEEATAEAYRYADALKKDNGEYTVDVADKGYTLNIKFA

SEQ ID NO: 14 - protein, 60 aa, Peptostreptococcus magnus
VTIKANLIYADGKTQTAEFKGTFEEATAEAYRYADLLAKENGKYTVDVADKGYTLNIKFA

SEQ ID NO: 15 - protein, 60 aa, Peptostreptococcus magnus
VTIKANLIYADGKTQTAEFKGTFAEATAEAYRYADLLAKENGKYTADLEDGGYTINIRFA

SEQ ID NO: 16 - protein, 60 aa, Peptostreptococcus magnus
VTIKANLIFADGSTQNAEFKGTFAKAVSDAYAYADALKKDNGEYTVDVADKGLTLNIKFA

SEQ ID NO: 17 - protein, 60 aa, Peptostreptococcus magnus
VTIKVNLIFADGKTQTAEFKGTFEEATAKAYAYADLLAKENGEYTADLEDGGNTINIKFA

SEQ ID NO: 18 - protein, 60 aa, Peptostreptococcus magnus
VTIKVNLIFADGKIQTAEFKGTFEEATAKAYAYANLLAKENGEYTADLEDGGNTINIKFA

SEQ ID NO: 19 - protein, 60 aa, Peptostreptococcus magnus
VTIKVNLIFADGKTQTAEFKGTFEEATAEAYRYADLLAKVNGEYTADLEDGGYTINIKFA

SEQ ID NO: 20 - protein, 60 aa, Artificial Sequence
  engineered PpL analogue
  MISC_FEATURE (5)..(5): Xaa at position 5 is Ala or Val;
  MISC_FEATURE (9)..(9): Xaa at position 9 is Phe or Tyr;
  MISC_FEATURE (11)..(11): Xaa at position 11 is Asp or Asn;
  MISC_FEATURE (13)..(13): Xaa at position 13 is Lys or Ser;
  MISC_FEATURE (14)..(14): Xaa at position 14 is Thr or Ile;
  MISC_FEATURE (16)..(16): Xaa at position 16 is Thr or Asn;
  MISC_FEATURE (24)..(24): Xaa at position 24 is Glu or Ala;
  MISC_FEATURE (25)..(25): Xaa at position 25 is Glu or Lys;
  MISC_FEATURE (27)..(27): Xaa at position 27 is Thr or Val;
  MISC_FEATURE (28)..(28): Xaa at position 28 is Ala or Ser;
  MISC_FEATURE (29)..(29): Xaa at position 29 is Glu, Lys or Asp;
  MISC_FEATURE (32)..(32): Xaa at position 32 is Arg or Ala;
  MISC_FEATURE (35)..(35): Xaa at position 35 is Asp or Asn;
  MISC_FEATURE (36)..(36): Xaa at position 36 is Leu, Ala or Thr;
  MISC_FEATURE (38)..(38): Xaa at position 38 is Ala or Lys;
  MISC_FEATURE (40)..(40): Xaa at position 40 is Glu, Asp or Val;
  MISC_FEATURE (43)..(43): Xaa at position 43 is Glu or Lys;
  MISC_FEATURE (46)..(46): Xaa at position 46 is Ala or Val;
  MISC_FEATURE (48)..(48): Xaa at position 48 is Leu or Val;
  MISC_FEATURE (49)..(49): Xaa at position 49 is Glu or Ala;
  MISC_FEATURE (51)..(51): Xaa at position 51 is Gly or Lys;
  MISC_FEATURE (53)..(53): Xaa at position 53 is Tyr, Asn or Leu;
  MISC_FEATURE (55)..(55): Xaa at position 55 is Ile or Leu;
  MISC_FEATURE (58)..(58): Xaa at position 58 is Lys or Arg;
VTIKXNLIXAXGXXQXAEFKGTFXXAXXXAYXYAXXLXKXNGXYTXDXXDXGXTXNIXFA

SEQ ID NO: 21 - DNA, 192 bp, Artificial Sequence
  DNA code
gaagaggttaccattaaagcgaacctgatctttgccaatggctcgacacagactgccgagttcaaaggtacgtttgagaaagctacttcagaagcttacgcctatgcggacacactcaagaaagacaatggcgaatacacagtggatgttgcagataaaggttacacactgaatattaaattcgccggctaa

SEQ ID NO: 22 - DNA, 66 bp, Artificial Sequence
  oligo DNA
cgtggatccgaagaggttaccattaaagcgaacctgatctttgccaatggctcgacacagactgcc

SEQ ID NO: 23 - DNA, 54 bp, Artificial Sequence
  oligo DNA
ggcgtaagcttctgaagtagctttctcaaacgtacctttgaactcggcagtctg

SEQ ID NO: 24 - DNA, 63 bp, Artificial Sequence
  oligo DNA
tcagaagcttacgcctatgcggacacactcaagaaagacaatggcgaatacacagtggatgtt

SEQ ID NO: 25 - DNA, 60 bp, Artificial Sequence
  oligo DNA
gatgaattcttagccggcgaatttaatattcagtgtgtaacctttatctgcaacatccac

SEQ ID NO: 26 - DNA, 48 bp, Artificial Sequence
  oligo DNA
tcagaagcttacgcctatgcggacacactcaagaaagacgctggcgaa

SEQ ID NO: 27 - DNA, 48 bp, Artificial Sequence
  oligo DNA
tcagaagcttacgcctatgcggacacactcaagaaagaccatggcgaa

SEQ ID NO: 28 - DNA, 51 bp, Artificial Sequence
  oligo DNA
tcagaagcttacgcctatgcggacacactcaagaaagacaatgccgaatac

SEQ ID NO: 29 - DNA, 192 bp, Artificial Sequence
  DNA code
gaagaggttaccattaaagcgaacctgatctttgccaatggctcgacacagactgccgagttcaaaggtacgtttgagaaagctacttcagaagcttacgcctatgcggacacactcaagaaagacgctggcgaatacacagtggatgttgcagataaaggttacacactgaatattaaattcgccggctaa

SEQ ID NO: 30 - DNA, 192 bp, Artificial Sequence
  DNA code
gaagaggttaccattaaagcgaacctgatctttgccaatggctcgacacagactgccgagttcaaaggtacgtttgagaaagctacttcagaagcttacgcctatgcggacacactcaagaaagaccatggcgaatacacagtggatgttgcagataaaggttacacactgaatattaaattcgccggctaa

SEQ ID NO: 31 - DNA, 192 bp, Artificial Sequence
  DNA code
gaagaggttaccattaaagcgaacctgatctttgccaatggctcgacacagactgccgagttcaaaggtacgtttgagaaagctacttcagaagcttacgcctatgcggacacactcaagaaagacaatgccgaatacacagtggatgttgcagataaaggttacacactgaatattaaattcgccggctaa

SEQ ID NO: 32 - protein, 60 aa, Artificial Sequence
  PpL mutatnt
VTIKANLIFANGSTQTAEFKGTFEKATSEAYAYADTLKKDAGEYTVDVADKGYTLNIKFA

SEQ ID NO: 33 - protein, 60 aa, Artificial Sequence
  PpL mutatnt
VTIKANLIFANGSTQTAEFKGTFEKATSEAYAYADTLKKDHGEYTVDVADKGYTLNIKFA

SEQ ID NO: 34 - protein, 60 aa, Artificial Sequence
  PpL mutatnt
VTIKANLIFANGSTQTAEFKGTFEKATSEAYAYADTLKKDNAEYTVDVADKGYTLNIKFA

SEQ ID NO: 35 - DNA, 39 bp, Artificial Sequence
  oligo DNA
cgtggatccgaagaggttaccattaaagcggccctgatc

SEQ ID NO: 36 - DNA, 54 bp, Artificial Sequence
  oligo DNA
cgtggatccgaagaggttaccattaaagcgaacctgatctttgccgctggctcg

SEQ ID NO: 37 - DNA, 57 bp, Artificial Sequence
  oligo DNA
cgtggatccgaagaggttaccattaaagcgaacctgatctttgccaatgcctcgaca

SEQ ID NO: 38 - DNA, 192 bp, Artificial Sequence
  DNA code
gaagaggttaccattaaagcggccctgatctttgccaatggctcgacacagactgccgagttcaaaggtacgtttgagaaagctacttcagaagcttacgcctatgcggacacactcaagaaagacaatggcgaatacacagtggatgttgcagataaaggttacacactgaatattaaattcgccggctaa

SEQ ID NO: 39 - DNA, 192 bp, Artificial Sequence
  DNA code
gaagaggttaccattaaagcgaacctgatctttgccgctggctcgacacagactgccgagttcaaaggtacgtttgagaaagctacttcagaagcttacgcctatgcggacacactcaagaaagacaatggcgaatacacagtggatgttgcagataaaggttacacactgaatattaaattcgccggctaa

SEQ ID NO: 40 - DNA, 192 bp, Artificial Sequence
  DNA code
gaagaggttaccattaaagcgaacctgatctttgccaatgcctcgacacagactgccgagttcaaaggtacgtttgagaaagctacttcagaagcttacgcctatgcggacacactcaagaaagacaatggcgaatacacagtggatgttgcagataaaggttacacactgaatattaaattcgccggctaa

SEQ ID NO: 41 - protein, 60 aa, Artificial Sequence
  PpL mutatnt
VTIKAALIFANGSTQTAEFKGTFEKATSEAYAYADTLKKDNGEYTVDVADKGYTLNIKFA

SEQ ID NO: 42 - protein, 60 aa, Artificial Sequence
  PpL mutatnt
VTIKANLIFAAGSTQTAEFKGTFEKATSEAYAYADTLKKDNGEYTVDVADKGYTLNIKFA

SEQ ID NO: 43 - protein, 60 aa, Artificial Sequence
  PpL mutatnt
VTIKANLIFANASTQTAEFKGTFEKATSEAYAYADTLKKDNGEYTVDVADKGYTLNIKFA

(19) United States
(12) Patent Application Publication
(10) Pub. No.: US 2019/0119333 A1
(43) Pub. Date: Apr. 25, 2019

(54) MODIFIED IMMUNOGLOBULIN K CHAIN VARIABLE REGION-BINDING PEPTIDE

(71) Applicant: KANEKA CORPORATION, Osaka (JP)

(72) Inventor: Shinichi Yoshida, Hyogo (JP)

(73) Assignee: KANEKA CORPORATION, Osaka (JP)

(21) Appl. No.: 16/176,364

(22) Filed: Oct. 31, 2018

Related U.S. Application Data
(63) Continuation of application No. PCT/JP2017/015501, filed on Apr. 17, 2017.

(30) Foreign Application Priority Data
May 2, 2016 (JP) 2016-092804

(51) Int. Cl. C07K 14/195; C07K 1/22

(52) U.S. Cl. CPC C07K 14/195; C07K 1/22

(57) ABSTRACT

A first immunoglobulin κ chain variable region-binding peptide includes an amino acid sequence of SEQ ID NO: 20 with at least one substitution at one or more positions selected from the group consisting of the 41^{st} position and the 42^{nd} position. A second immunoglobulin κ chain variable region-binding peptide includes the amino acid sequence further comprising 1 to 20 amino acid deletions, substitutions and/or additions at one or more positions except for the 41^{st} position and the 42^{nd} position. A third immunoglobulin κ chain variable region-binding peptide includes an amino acid sequence having a sequence identity of 80% or more with the amino acid sequence of the first peptide, provided that the at least one substitution is not further mutated. The second and third peptides have a higher chemical stability to an alkaline aqueous solution than the chemical stability of the first peptide before introducing the substitution.

### TECHNICAL FIELD

One or more embodiments of the present invention relate to an immunoglobulin κ chain variable region-binding peptide of which chemical stability to an alkaline solution is improved, an affinity separation matrix which has the peptide as a ligand, a method for producing an immunoglobulin κ chain variable region-containing protein by using the affinity separation matrix, a DNA which encodes the peptide, a vector which contains the DNA, and a transformant which is 16 transformed by the vector.

### BACKGROUND

As one of important functions of a protein, an ability to specifically bind to a specific molecule is exemplified. The function plays an important role in an immunoreaction and signal transduction in a living body. A technology utilizing the function for purifying a useful substance has been actively developed. As one example of proteins which are actually utilized industrially, for example, Protein A affinity separation matrix has been used for capturing an antibody drug to be purified with high purity at one time from a culture of an animal cell (Non-patent documents 1 and 2). Hereinafter, Protein A is abbreviated as “SpA” in some cases.

An antibody drug which has been developed is mainly a monoclonal antibody, and a monoclonal antibody has been produced on a large scale by using recombinant cell cultivation technology. A “monoclonal antibody” means an antibody obtained from a clone derived from a single antibody-producing cell. Most of antibody drugs which are presently launched are classified into an immunoglobulin G (IgG) subclass in terms of a molecular structure. In addition, an antibody drug consisting of an antibody fragment has been actively subjected to clinical development. An antibody fragment has a molecular structure obtained by fragmenting an immunoglobulin, and various antibody fragment drugs have been clinically developed (Non-patent Document 3).

In an initial purification step of an antibody drug production process, the above-described SpA affinity separation matrix is utilized. SpA is, however, basically a protein which specifically binds to a Fc region of IgG. Thus, SpA affinity separation matrix cannot capture an antibody fragment which does not contain a Fc region. Accordingly, an affinity separation matrix capable of capturing an antibody fragment which does not contain a Fc region of IgG is highly required industrially in terms of a platform development of a process for purifying an antibody drug.

A plurality of peptides which bind to a region except for a Fc region of IgG have been already known (Non-patent Document 4). Among such peptides, a peptide which can bind to a variable region as an antibody-binding domain is most preferred in terms of many kinds of antibody fragment format to be bound and an ability to bind to IgM and IgA. As such a peptide, for example, Protein L has been well-known. Hereinafter, Protein L is abbreviated as “PpL” in some cases. PpL is a protein which contains a plurality of κ-chain variable region-binding domains, and amino acid sequences of each κ-chain variable region-binding domain are different from each other. Hereinafter, κ-chain variable region is abbreviated as “VL-κ” in some cases. In addition, the number of VL-κ-binding domains and amino acid sequences of each VL-κ-binding domain are different depending on the kind of a strain. For example, the number of VL-κ-binding domains in PpL of Peptostreptococcus magnus 312 strain is 5, and the number of VL-κ-binding domains in PpL of Peptostreptococcus magnus 3316 strain is 4 (Non-patent documents 5 to 7, and Patent documents 1 and 2). There are no domains that have the same amino acid sequence as each other in the totally 9 VL-κ-binding domains.

In the case of SpA, a protein engineering study to site-specifically introduce a mutation to improve a function as a ligand for an affinity separation matrix has been actively promoted (Non-patent documents 1 and 8, and Patent documents 3 to 8). In particular, many of them are researches aimed at improving the chemical stability of SpA against sodium hydroxide solution used for washing SpA affinity separation matrix. Specifically, it shows an affect to improve the chemical stability to substitute an asparagine residue and substitute a glycine residue after an asparagine residue, since it has been known that an asparagine residue is susceptible to a deamidation reaction under an alkaline condition. However, with respect to all of asparagine residues in SpA, the improvement effect is not always shown (Non-patent document 8).

A plurality of affinity separation matrixes having PpL as a ligand have been commercially available. Various introduction of a mutation to evaluate a binding strength and a binding mode of PpL have been reported (Non-patent documents 7, 9 and 10). A study to modify a function of PpL as an affinity ligand has also been reported (Patent document 9). However, the report number about a mutation introduction to PpL is smaller than that of SpA. In particular, with respect to a chemical stability of SpA affinity separation matrix to an alkaline solution, the matrix can be washed with 0.1 to 0.5 M sodium hydroxide by the improvement of SpA. On the one hand, in the case of PpL, 0.02 to 0.05 M sodium hydroxide is recommended (Non-patent document 11). Thus, in the case of PpL, there remains room for improvement on a chemical stability to an alkaline solution.

### PATENT DOCUMENT

Patent Document 1: JP H7-506573 T
Patent Document 2: JP H7-507682 T
Patent Document 3: U.S. Pat. No. 5,143,844 B
Patent Document 4: JP 2006-304633 A
Patent Document 5: EP 1123389 A
Patent Document 6: WO 03/080655
Patent Document 7: US 2006/0194950 A
Patent Document 8: WO 2011/118699
Patent Document 9: WO 00/15803

### Non-Patent Document

Non-patent Document 1: Hober S., et al., J. Chromatogr. B, 2007, vol. 848, pp. 40-47
Non-patent Document 2: Shukla A. A., et al., Trends Biotechnol., 2010, vol. 28, pp. 253-261
Non-patent Document 3: Nelson A. N., et al., Nat. Biotechnol., 2009, vol. 27, pp. 331-337
Non-patent Document 4: Bouvet P. J., Int. J. Immunopharmac., 1994, vol. 16, pp. 419-424
Non-patent Document 5: Kastern W., et al., J. Biol. Chem., 1992, vol. 267, pp. 12820-12825
Non-patent Document 6: Murphy J. P., et al., Mol. Microbiol., 1994, vol. 12, pp. 911-920
Non-patent Document 7: Housden N. G., et al., Biochemical Society Transactions, 2003, vol. 31, pp. 716-718
Non-patent Document 8: Linhult M., et al., PROTEINS, 2004, vol. 55, pp. 407-416
Non-patent Document 9: Housden N. G., et al., J. Biol. Chem., 2004, vol. 279, pp. 9370-9378
Non-patent Document 10: Tadeo X., et al., Biophys. J., 2009, vol. 97, pp. 2595-2603
Non-patent Document 11: Rodrigo G., et al., Antibodies, 2015, vol. 4, pp. 259-277

### SUMMARY

One or more embodiments of the present invention provide a novel modified Protein L (PpL) which can bind to K chain of an immunoglobulin and which is excellent in the chemical stability to an alkaline solution, an affinity separation matrix having the modified PpL as a ligand, and a method for producing a K chain variable region-containing protein by using the affinity separation matrix.

The inventor designed a molecule of a mutant of a VL-κ-binding domain of PpL, obtained the mutant from a transformed cell by using a protein engineering method and a genetic engineering method, and compared the properties of the obtained mutants.

Hereinafter, one or more embodiments of the present invention are described.

[1] An immunoglobulin κ chain variable region-binding peptide selected from the following (1) to (3):

(1) an immunoglobulin κ chain variable region-binding peptide 16 having an amino acid sequence of SEQ ID NO: 20 with substitution at one or more positions selected from the 41^{st} position and the 42^{nd} position (“first peptide”);

(2) an immunoglobulin κ chain variable region-binding peptide having an amino acid sequence specified in the (1) with deletion, substitution and/or addition of 1 or more and 20 or less amino acid residues in a region except for the 41^{st} position and the 42^{nd} position, and having a higher chemical stability to an alkaline aqueous solution than that before introducing the substitution specified in the (1) (“second peptide”);

(3) an immunoglobulin κ chain variable region-binding peptide having an amino acid sequence having a sequence identity of 80% or more with the amino acid sequence specified in the (1), and having a higher chemical stability to an alkaline aqueous solution than that before introducing the substitution specified in the (1), provided that the amino acid residue substitution at the one or more positions selected from the 41^{st} position and the 42^{nd} position specified in the (1) is not further mutated in (3) (“third peptide”).

[2] The immunoglobulin κ chain variable region-binding peptide according to the above [1], wherein the amino acid sequence specified in the (1) is any one of amino acid sequences of SEQ ID NOs: 12 to 19.

[3] The immunoglobulin κ chain variable region-binding peptide according to the above [1], wherein the amino acid residue at the 41^{st} position is substituted in the amino acid sequence specified in the (1).

[4] The immunoglobulin κ chain variable region-binding peptide according to any one of the above [1] to [3], wherein the amino acid residue at the 41^{st} position is substituted by Ala or His and the amino acid residue at the 42^{nd} position is substituted by A1a in the amino acid sequence specified in the (1).

[5] The immunoglobulin κ chain variable region-binding peptide according to any one of the above [1] to [4], wherein a position of the deletion, substitution and/or addition is N-terminal and/or C-terminal in the amino acid sequence specified in the (2).

[6] The immunoglobulin κ chain variable region-binding peptide according to any one of the above [1] to [5], wherein the sequence identity is 95% or more in the amino acid sequence specified in the (3).

[7] An immunoglobulin κ chain variable region-binding peptide multimer, comprising the two or more immunoglobulin κ chain variable region-binding peptides according to any one of the above [1] to [6] as domains, wherein the immunoglobulin κ chain variable region-binding peptides are connected one another.

[8] An affinity separation matrix, wherein the immunoglobulin κ chain variable region-binding peptide according to any one of the above [1] to [6] or the immunoglobulin κ chain variable region-binding peptide multimer according to the above [7] is immobilized as a ligand on a water-insoluble carrier.

[9] A method for producing a protein comprising a immunoglobulin κ chain variable region, comprising the steps of:

contacting a liquid sample comprising the protein comprising the immunoglobulin κ chain variable region with the affinity separation matrix according to the above [8]; and

separating the protein comprising the immunoglobulin κ chain variable region adsorbed on the affinity separation matrix from the affinity separation matrix.

[10] A DNA, encoding the immunoglobulin κ chain variable region-binding peptide according to any one of the above [1] to [6] or the immunoglobulin κ chain variable region-binding peptide multimer according to the above [7].

[11] A vector, comprising the DNA according to the above [10].

[12] A transformant, transformed by the vector according to the above [11].

The antibody κ chain-binding activity of the affinity chromatography carrier for purification on which the modified PpL of one or more embodiments of the present invention is immobilized is hardly decreased due to damage by an alkaline treatment. Thus, when the carrier is repeatedly used, the carrier can be washed by using a sodium hydroxide aqueous solution having a high concentration for a long time. As a result, an impurity such as an organic compound remaining in 16 the chromatography carrier can be effectively removed.

### BRIEF DESCRIPTION OF THE DRAWINGS

FIG. 1 represents an alignment of amino acid sequences of VL-κ-binding domains derived from PpL.

FIG. 2 is a figure to show a method for producing an expression vector of LB1t-Wild.1d.

FIGS. 3A to 3C are figures to show methods for producing expression vectors of various modified LB1t.

FIG. 4 is a graph in which binding responses to evaluate aHER-Fab-binding residual activity of various modified LB1t at various peptide concentrations.

FIG. 5 is a graph to show aHER-Fab-binding residual activities of various modified LB1t after an alkaline treatment.

FIG. 6 is a graph to show aIgE-Fab-binding residual activities of various modified LB1t after an alkaline treatment.

### DETAILED DESCRIPTION OF THE EMBODIMENTS

One or more embodiments of the present invention relate to the immunoglobulin κ chain variable region-binding peptide selected from the following (1) to (3):

(1) an immunoglobulin κ chain variable region-binding peptide having an amino acid sequence of SEQ ID NO: 20 with substitution at one or more positions selected from the 41^{st} position and the 42^{nd} position;

(2) an immunoglobulin κ chain variable region-binding peptide 16 having an amino acid sequence specified in the (1) with deletion, substitution and/or addition of one or more and 20 or less amino acid residues in a region except for the 41^{st} position and the 42^{nd} position, and having a higher chemical stability to an alkaline aqueous solution than that before introducing the substitution specified in the (1);

(3) an immunoglobulin κ chain variable region-binding peptide having an amino acid sequence having a sequence identity of 80% or more with the amino acid sequence specified in the (1), and having a higher chemical stability to an alkaline aqueous solution than that before introducing the substitution specified in the (1), provided that the amino acid residue substitution at one or more positions selected from the 41^{st} position and the 42^{nd} position is not further mutated in (3)

An “immunoglobulin (Ig)” is a glycoprotein produced by a B cell of a lymphocyte and has a function to recognize a specific molecule such as a protein to be bound. An immunoglobulin has not only a function to specifically bind to a specific molecule referred to as antigen but also a function to detoxify and remove an antigen-containing factor in cooperation with other biological molecule or cell. An immunoglobulin is generally referred to as “antibody”, and the name is inspired by such functions.

All of immunoglobulins basically have the same molecular structure. The basic structure of an immunoglobulin is a Y-shaped four-chain structure. The four-chain structure is composed of two light chains and two heavy chains of polypeptide chains. A light chain (L chain) is classified into two types of A chain and K chain, and all of immunoglobulins have either of the chains. A heavy chain (H chain) is classified into five types of γ chain, μ chain, α chain, δ chain and ε chain, and an immunoglobulin is classified into isotypes depending on the kind of a heavy chain. An immunoglobulin G (IgG) is a monomer immunoglobulin, is composed of two γ chains and two light chains, and has two antigen-binding sites.

A lower half vertical part in the “Y” shape of an immunoglobulin is referred to as a “Fc region”, and an upper half “V” shaped part is referred to as a “Fab region”. A Fc region has an effector function to initiate a reaction after an antibody binds to an antigen, and a Fab region has a function to bind to an antigen. A Fab region of a heavy chain and a Fc region are bound to each other through a hinge part. Papain, which is a proteolytic enzyme and which is contained in papaya, decomposes a hinge part to cut into two Fab regions and one Fc region. The part close to the tip of the “Y” shape in a Fab region is referred to as a “variable region (V region)”, since there are various changes of the amino acid sequence in order to bind to various antigens. A variable region of a light chain is referred to as a “VL region”, and a variable region of a heavy chain is referred to as a “VH region”. A Fab region except for a V region and a Fc region are referred to as a “constant region (C region)”, since there is relatively less change. A constant region of a light chain is referred to as a “CL region”, and a constant region of a heavy chain is referred to as a “CH region”. A CH region is further classified into three regions of CH1 to CH3. A Fab 16 region of a heavy chain is composed of a VH region and CH1, and a Fc region of a heavy chain is composed of CH2 and CH3. There is a hinge part between CH1 and CH2. Protein L binds to a variable region of which light chain is K chain (VL-κ) (Non-patent Documents 5 to 7).

The peptide of one or more embodiments of the present invention binds to a K chain variable region of an immunoglobulin. The K chain variable region is abbreviated as “VL-κ” in some cases. The VL-κ-containing protein to which the peptide of one or more embodiments of the present invention binds may contain VL-κ, and may be IgG containing a Fab region and a Fc region without deficiency, or a Ig derivative such as IgM, IgD and IgA, or a derivative of an immunoglobulin molecule prepared by a protein engineering mutation. An immunoglobulin molecule derivative to which the VL-κ-binding peptide of one or more embodiments of the present invention binds is not particularly restricted as long as the derivative contains VL-κ. For example, the immunoglobulin molecule derivative is exemplified by a Fab fragment prepared by fragmenting immunoglobulin G into a Fab region only, scFv consisting of a variable region of immunoglobulin G, chimeric immunoglobulin G prepared by replacing a part of human immunoglobulin G domains with an immunoglobulin G domain of other organism to be fused, immunoglobulin G of which sugar chain in the Fc region is mutated, and a scFv fragment to which a drug is covalently bonded.

The term “peptide” in one or more embodiments of the present invention means any molecules having a polypeptide 16 structure. In the range of the “peptide”, not only a so-called protein but also a fragmented protein and a protein to which other peptide is bound through a peptide bond are included. In one or more embodiments of the present invention, a peptide is basically used synonymously with a protein.

The term “domain” means a unit of higher-order structure of a protein. A domain is composed of from dozens to hundreds of amino acid residues, and means a protein unit which can sufficiently serve some kind of a physicochemical or biochemical function.

The term “mutant” of a protein or peptide means a protein or peptide obtained by introducing at least one substitution, addition or deletion of an amino acid into a sequence of a wild protein or peptide. A mutation to substitute an amino acid is described by adding a wild or non-mutated amino acid residue before the number of a substituted position and adding a mutated amino acid residue after the number of the substituted position. For example, the mutation to substitute Gly at the 290 position by A1a is described as G29A.

The “Protein L” (PpL) is a protein derived from a cell wall of anaerobic gram-positive coccus in the genus of Peptostreptococcus. In one or more embodiments, PpL is preferably derived from Peptostreptococcus magnus, and preferably 2 kinds of PpL derived from Peptostreptococcus magnus 312 strain and Peptostreptococcus magnus 3316 strain, but is not restricted thereto (Non-patent documents 4 to 6). In one or more embodiments of the present invention, in some 16 cases, the PpL derived from Peptostreptococcus magnus 312 strain is abbreviated as “PpL 312”, and the PpL derived from Peptostreptococcus magnus 3316 strain is abbreviated as “PpL 3316”. The amino acid sequence of PpL 312 is shown as SEQ ID NO: 1, and the amino acid sequence of PpL 3316 is shown as SEQ ID NO: 2, which also contain a signal sequence.

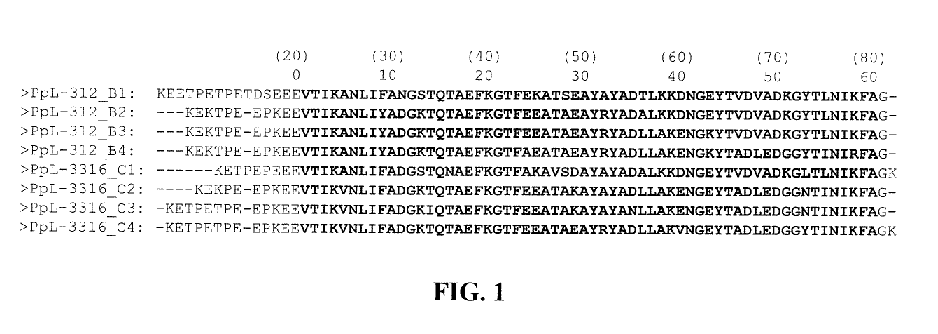

PpL contains a plurality of VL-κ-binding domains having 70 to 80 residues. The number of VL-κ-binding domains contained in PpL 312 is 5, and the number of VL-κ-binding domains contained in PpL 3316 is 4. The VL-κ-binding domains contained in PpL 312 are referred to as B1 domain (SEQ ID NO: 3), B2 domain (SEQ ID NO: 4), B3 domain (SEQ ID NO: 5), B4 domain (SEQ ID NO: 6), and B5 domain (SEQ ID NO: 7) in the order from the N-terminal, and the VL-κ-binding domains contained in PpL 3316 are referred to as C1 domain (SEQ ID NO: 8), C2 domain (SEQ ID NO: 9), C3 domain (SEQ ID NO: 10), and C4 domain (SEQ ID NO: 11) in the order from the N-terminal (Non-patent documents 5 and 6). From the contents of Patent documents 1 and 2, it is preferred that one or more embodiments of the present invention are applied to B1, B2, B3, B4, C1, C2, C3, or C4 domain, of which usability is demonstrated by Example or the like. The alignment of amino acid sequences of the VL-κ-binding domains is demonstrated as FIG. 1. In FIG. 1, the residue number identified in accordance with Non-patent Documents 7 and 8 and Patent Document 9 is described in the parentheses.

It has been found from a research that about 20 residues of the VL-κ-binding domains at the N-terminal part do not form 16 a specific secondary structure; and even when the N-terminal region is deleted, the three-dimensional structure and the VL-K-binding property of a VL-κ-binding domain are maintained (Non-patent Document 7). As a result, for example, peptides having the amino acid sequence of SEQ ID NO: 12 with respect to B1 domain, the amino acid sequence of SEQ ID NO: 13 with respect to B2 domain, the amino acid sequence of SEQ ID NO: 14 with respect to B3 domain, the amino acid sequence of SEQ ID NO: 15 with respect to B4 domain, the amino acid sequence of SEQ ID NO: 16 with respect to C1 domain, the amino acid sequence of SEQ ID NO: 17 with respect to C2 domain, the amino acid sequence of SEQ ID NO: 18 with respect to C3 domain, and the amino acid sequence of SEQ ID NO: 19 with respect to C4 domain also function as a VL-κ-binding domain. It is further preferred that the amino acid sequence used in one or more embodiments of the present invention is the amino acid sequence of SEQ ID NO: 20, which exhaustively contains the amino acid residues common to the above-described domains (SEQ ID NOs: 12 to 19). In one or more embodiments of the present invention, an amino acid residue number is given in accordance with a definition that the N-terminal residue of SEQ ID NO: 20 is the 1^{st} position. In FIG. 1, residue numbers are described in accordance with the above-described definition, and the residues from Val at the 1^{st} position through A1a at the 60^{th} position of the above-described domains (SEQ ID NOs: 12 to 19) are described in boldface.

In one or more embodiments of the present invention, the specific substitution mutation is introduced in the amino acid sequence of VL-κ-binding domain of wild PpL; as a result, the 16 chemical stability of the mutant to an alkaline aqueous solution is improved in comparison with that before introducing the mutation.

As experimentally demonstrated in Examples described later, even when the modified VL-κ-binding peptide (1) is treated by an alkaline aqueous solution, the damage due to alkali is smaller and the binding ability to VL-K is maintained at high level.

The substitution position of the modified VL-κ-binding peptide (1) is one or more amino acid residues at the positions selected from the 41^{st} position and the 42^{nd} position of the amino acid sequence of SEQ ID NO: 20. The 41^{st} position of SEQ ID NO: 20 is Asn, and the 42^{nd} position is Gly. Even when the numbers of amino acids in the amino acid sequences before and after the introduction of the mutation are different, a skilled person can easily identify the position corresponding to the 41^{st} position and the 42^{nd} position of SEQ ID NO: 20 under the condition that the number of the modified amino acid is not more than 20 or the sequence identity is 80% or more. Specifically, the position can be confirmed by aligning the sequences with using a program for amino acid sequence multiple alignment: Clustal (http://www.clustal.org/omega/) or gene information processing software: GENETYX (https://www.genetyx.co.jp/). The substitution position of the amino acid residue in one or more embodiments of the present invention corresponds to the 61^{st} position and the 62^{nd} position on the basis of the residue number described in Non-patent Documents 7 and 8 and Patent 16 Document 9.

The VL-κ-binding peptide (1) of one or more embodiments of the present invention has the amino acid sequence in which one or more amino acid residues at positions selected from the 41^{st} position and the 42^{nd} position of SEQ ID NO: 20 are substituted. In one or more embodiments, the substitution position is preferably the 41^{st} position.

In one or more embodiments of the present invention, the phrase, a peptide “has a (specific) amino acid sequence”, means that the specific amino acid sequence is contained in the amino acid sequence of the peptide and the function of the peptide is maintained. The amino acid sequence of the peptide may be the same as a specific amino acid sequence itself, or a sequence of a specific amino sequence bound by other amino acid sequence. The sequence of the peptide other than a specific amino acid sequence is exemplified by histidine tag, a linker sequence for immobilization, and a crosslinking structure such as —S—S— bond.

In one or more embodiments, the kind of an amino acid for mutation is not particularly restricted, the mutation may be a substitution by a non-protein-constituting amino acid and a non-natural amino acid, and a natural amino acid can be preferably used in terms of genetic engineering production. A natural amino acid is classified into the categories of a neutral amino acid; an acidic amino acid such as Asp and Glu; and a basic amino acid such as Lys, Arg and His. A neutral amino acid is classified into the categories of an aliphatic 16 amino acid; an imino acid such as Pro; and an aromatic amino acid such as Phe, Tyr and Trp. An aliphatic amino acid is classified into the categories of Gly; Ala; a branched amino acid such as Val, Leu and Ile; a hydroxy amino acid such as Ser and Thr; a sulfur-containing amino acid such as Cys and Met; and an acid amide amino acid such as Asn and Gln. Since Tyr has a phenolic hydroxyl group, Tyr may be classified into not only an aromatic amino acid but also a hydroxy amino acid. From another viewpoint, a natural amino acid may also be classified into the categories of a nonpolar amino acid with high hydrophobicity, such as Gly, Ala, Val, Leu, Ile, Trp, Cys, Met, Pro and Phe; a neutral polar amino acid such as Asn, Gln, Ser, Thr and Tyr; an acidic polar amino acid such as Asp and Glu; and a basic polar amino acid such as Lys, Arg and His.

In the amino acid sequence of SEQ ID NO: 20, the amino acid at the 41^{st} position is Asn and the amino acid at the 42^{nd} position is Gly. In one or more embodiments of the present invention, for example, the amino acid at the 41^{st} position is preferably substituted by an amino acid except for an acid amide amino acid, more preferably an aliphatic amino acid, a nonpolar amino acid or a basic amino acid, more preferably Ala or His, and more preferably His. For example, the amino acid at the 42^{nd} position is preferably substituted by an aliphatic amino acid, and more preferably Ala.

The modified VL-κ-binding peptide (2) is an immunoglobulin κ chain variable region-binding peptide which has the amino 16 acid sequence specified in the above-described (1) with deletion, substitution and/or addition of 1 or more and 20 or less amino acid residues in a region except for the 41^{st} position and the 42^{nd} position, wherein the chemical stability thereof to an alkaline aqueous solution is higher than that before introducing the substitution specified in the modified VL-κ-binding peptide (1).

In one or more embodiments, the number of the mutation of the above-described deletion, substitution and/or addition is preferably not more than 15 or not more than 10, more preferably not more than 7, not more than 5 or not more than 3, even more preferably 1 or 2, and particularly preferably 1. In the amino acid sequence of the modified VL-κ-binding peptide (2) according to one or more embodiments of the present invention, the position of the deletion, substitution and/or addition of the amino acid residue is not particularly restricted as long as the position is not the 41^{st} position and the 42^{nd} position specified in the VL-κ-binding peptide (1). The position of the deletion, substitution and/or addition of the amino acid residue is exemplified by N-terminal and/or C-terminal. In one or more embodiments, the terminal positions are particularly preferred as the position of the deletion and/or addition.

The amino acid sequences of SEQ ID NOs: 12 to 19 corresponds to the amino acid sequences of SEQ ID NOs: 3 to 6 or SEQ ID NOs: 8 to 11 with deletion of 10 to 20 residues at the N-terminal and 1 to 2 residues at the C-terminal. Accordingly, as one embodiment, the amino acid sequence to be added to the N-terminal and/or C-terminal is exemplified by the above-described amino acid sequence at the N-terminal and/or C-terminal. As one embodiment, the amino acid sequence to be added to the N-terminal is exemplified by Glu-Glu or Glu-Gln. As one embodiment, the amino acid sequence to be added to the C-terminal is exemplified by Gly, Cys or Gly-Cys.

The modified VL-κ-binding peptide (3) is an immunoglobulin κ chain variable region-binding peptide which has an amino acid sequence with a sequence identity of 80% or more to the amino acid sequence specified in the above-described (1), wherein the chemical stability thereof to an alkaline aqueous solution is higher than that before introducing the substitution specified in the modified VL-κ-binding peptide (1), provided that the amino acid residue substitution at the one or more positions selected from the 41^{st} position and the 42^{nd} position specified in the above-described (1) is not further mutated in (3).

In one or more embodiments, the above-described sequence identity is preferably 85% or more, more preferably not less than 90%, not less than 95%, not less than 98% or not less than 99%, and particularly preferably 99.5% or more. The sequence identity can be evaluated by a program for amino acid sequence multiple alignment, such as Clustal (http://www.clustal.org/omega/), as described above.

The VL-κ-binding peptides (1) to (3) of one or more embodiments of the present invention are characterized in that the chemical stability thereof to an alkaline aqueous solution is improved in comparison with that before introducing the substitution. In other words, the chemical stability of the VL-κ-binding peptides (1) to (3) according to one or more embodiments of the present invention is improved in comparison with than that of the peptide having the amino acid sequence of SEQ ID NO: 20.

The “alkaline aqueous solution” shows alkalinity to the degree that the solution can attain the object of cleaning or sterilization. More specifically, sodium hydroxide aqueous solution of not less than 0.01 M and not more than 1.0 M or not less than 0.01 N and not more than 1.0 N can be used as the alkaline aqueous solution, but the solution is not restricted thereto. In one or more embodiments, in the case of sodium hydroxide, the lower limit of the concentration is preferably 0.01 M, more preferably 0.02 M, and even more preferably 0.05 M. On the one hand, the upper limit of sodium hydroxide concentration is preferably 1.0 M, more preferably 0.5 M, even more preferably 0.3 M, even more preferably 0.2 M, and even more preferably 0.1 M. The alkaline aqueous solution is not necessarily a sodium hydroxide aqueous solution, and in one or more embodiments, the pH thereof is preferably 12 or more and 14 or less. In one or more embodiments, with respect to the lower limit of the pH, 12.0 or more is preferred, and 12.5 or more is more preferred. With respect to the upper limit of the pH, 14 or less is preferred, 13.5 or less is more preferred, and 13.0 or less is even more preferred.

The term “chemical stability” means the property that a protein retains the function against a chemical modification such as a chemical change of an amino acid residue and chemical denaturation such as transposition and cleavage of an amide bond. In one or more embodiments of the present invention, the function of the peptide means a binding activity to VL-κ. In one or more embodiments of the present invention, a “binding activity to VL-κ” means a ratio of a polypeptide which does not undergo chemical denaturation and which retains an affinity for VL-κ. Thus, when the “chemical stability” is higher, the degree to decrease the binding activity to VL-κ after an immersion treatment in an alkaline aqueous solution is smaller. In one or more embodiments of the present invention, the term “resistance to alkali” has the same meaning as “chemical stability under an alkaline condition”.

The time to immerse the peptide in alkali is not particularly restricted, since a damage degree of the peptide is different depending on the concentration of the alkali and the temperature at the immersion. In one or more embodiments, for example, when the concentration of sodium hydroxide is 0.05 M and the temperature during immersion is atmospheric temperature, the lower limit of the time for an immersion in an alkali is preferably 1 hour, more preferably 2 hours, more preferably 4 hours, more preferably 10 hour, and more preferably 20 hours, but is not particularly restricted.

The affinity for an immunoglobulin can be evaluated by a biosensor such as Biacore system (GE Healthcare) utilizing a surface plasmon resonance principle, but the means is not restricted thereto. The measurement condition may be adjusted so that when the peptide of one or more embodiments of the present invention binds to VL-κ, a binding signal can be detected. In one or more embodiments, with respect to the measurement condition, it is preferred that the temperature is kept in the range of 20° C. or higher and 40° C. or lower, and the pH when the binding condition is observed is adjusted to be neutral such as about 5 or more and 8 or less. A component of a buffer solution is exemplified by phosphoric acid, tris(hydroxymethyl)aminomethane, bis[tris(hydroxymethyl)aminomethane] in the case of a neutral buffer, but is not restricted thereto. The concentration of sodium chloride in a buffer solution is not particularly restricted, and in one or more embodiments, it is preferably about 0 M more and 0.15 M or less.

A parameter as an indicator of binding to VL-κ is exemplified by an affinity constant (K_{A}) and a dissociation constant (K_{D}) (Nagata et al., “Real-time analysis experiment of biomaterial interactions”, Springer-Verlag Tokyo, 1998, page 41). An affinity constant of the peptide according to one or more embodiments to VL-κ can be measured by using Biacore system, specifically by immobilizing a human IgG on a sensor tip and adding each modified domain to be flown into a channel in the condition of 25° C. and pH 7.4. In one or more embodiments, an affinity constant K_{A} of the present invention protein to human VL-κ is preferably 1×10 M⁻¹ or more, more preferably 1×10⁶ M⁻¹ or more; however, such an affinity constant is not restricted to the above-described values, since an affinity constant is varied depending on the kind of a VL-κ-containing peptide and the number of domains of VL-κ-binding peptide.

When a residual binding activity after an alkaline treatment is determined, K_{A} and K_{D} are inappropriate as binding parameters. This is because even if the ratio of the molecule capable of binding to VL-κ changes by an alkaline treatment, change of K_{A} and K_{D} is not observed as a parameter when the binding ability to VL-κ of one peptide molecule does not change. In one or more embodiments, for example, in order to determine a residual binding activity of the peptide, it is preferred that VL-κ is immobilized on a sensor chip, and the degree of a binding signal or a theoretical maximum binding capacity (Rₘₐₓ) at the time of the addition of an immunoglobulin in the same concentrations before and after the peptide is chemically treated is used as the binding parameter, but the binding parameter is not restricted thereto. The theoretical maximum binding capacity (Rₘₐₓ) is a binding parameter of which unit is resonance unit (RU) to indicate the magnitude of a binding response. For example, the peptide is immobilized, VL-κ-containing peptides are added in the same concentrations before and after the immobilized chip is subjected to an alkaline treatment, a binding signal is measured before and after the alkaline treatment, and the binding signals may be compared.

Since a residual binding activity is determined by comparing the data before and after an alkaline treatment, a residual binding activity can be basically represented as a ratio (percentage) of a binding activity after an alkaline treatment as a numerator to a binding activity before the alkaline treatment as a denominator. The value of a residual binding activity is not particularly restricted as long as the value is higher than that of the peptide in which the mutation of one or more embodiments of the present invention is not introduced and which is subjected to the alkaline treatment in the same condition, and in one or more embodiments, it is preferably 10% or more, more preferably 20% or more, even more preferably 30% or more, even more preferably 40% or more, and even more preferably 50% or more.

It is important that the sample as a comparative control is the same except that the mutation of one or more embodiments of the present invention is not introduced, the amino acid sequence is the same, and all of the condition of the alkaline treatment and the measurement condition of a residual binding activity are the same. In addition, since the peptide of one or more embodiments does not exhibit a VL-κ-binding activity in an alkaline aqueous solution, an appropriate treatment is needed. For example, pH after the alkaline treatment is adjusted to be neutral by using an acid.

Protein L (PpL) is a protein in which 4 or 5 VL-κ-binding domains are linked in tandem. The VL-κ-binding peptide of one or more embodiments of the present invention, therefore, may be a monomer or a multimer composed of the 2 or more, preferably 3 or more, even more preferably 4 or more, and even more preferably 5 or more linked VL-κ-binding peptides as a monodomain. With respect to the upper limit of the number of the domains to be linked, 10 or less is exemplified, 8 or less is preferred, and 6 or less is more preferred. The multimer may be a homomultimer in which one kind of the VL-κ-binding peptides are linked, such as homodimer and homotrimer, or a heteromultimer in which two or more kinds of the VL-κ-binding peptides are linked, such as heterodimer and heterotrimer.

A method for connection to form the VL-κ-binding peptide multimer of one or more embodiments of the present invention is exemplified by a connecting method through one or more amino acid residues and a direct connecting method without an amino acid residue, but is not restricted thereto. The number of the amino acid residue for connection is not particularly restricted, and in one or more embodiments, it is preferably 20 residues or less, more preferably 15 residues or less, even more preferably 10 or less, even more preferably 5 or less, and even more preferably 2 or less. In one or more embodiments, it is preferred that the amino acid residue for connection does not destabilize a three dimensional structure of the monomer protein.

As one of the embodiments, a fusion peptide characterized in that the VL-κ-binding peptide of one or more embodiments of the present invention or a multimer thereof is fused as one component with other peptide having a different function is exemplified. Such a fusion peptide is exemplified by a peptide fused with albumin or GST, i.e. glutathione S-transferase, but is not restricted to the examples. In addition, peptides fused with a nucleic acid such as DNA aptamer, a drug such as an antibiotic or a polymer such as PEG, i.e. polyethylene glycol, are also included in the range of one or more embodiments of the present invention as long as the availability of the peptide according to the peptide of one or more embodiments is utilized in such a fusion peptide.

It is included in the present disclosure as one embodiment that the peptide of one or more embodiments is utilized as an affinity ligand having an affinity for an immunoglobulin or a fragment thereof, particularly VL-κ. An affinity separation matrix prepared by immobilizing the ligand on a water-insoluble carrier is similarly included in the present disclosure as one embodiment.

The affinity separation matrix of one or more embodiments of the present invention is characterized by immobilizing the above-described immunoglobulin κ chain variable region-binding peptide or the immunoglobulin κ chain variable region-binding peptide multimer according to one or more embodiments of the present invention as a ligand on a water-insoluble carrier.

The term “ligand” in one or more embodiments of the present invention means a substance and a functional group to selectively capture or bind to a target molecule from an aggregate of molecules on the basis of a specific affinity between molecules, such as binding between an antigen and an antibody, and means the peptide which specifically binds to an immunoglobulin in one or more embodiments of the present invention. In one or more embodiments of the present invention, the term “ligand” also means an “affinity ligand”.

The water-insoluble carrier usable in one or more embodiments of the present invention is exemplified by an inorganic carrier such as glass beads and silica gel; an organic carrier composed of a synthetic polymer such as cross-linked polyvinyl alcohol, cross-linked polyacrylate, cross-linked polyacrylamide and cross-linked polystyrene; an organic carrier composed of a polysaccharide such as crystalline cellulose, cross-linked cellulose, cross-linked agarose and cross-linked dextran; and a composite carrier obtained by the combination of the above carriers, such as an organic-organic composite carrier and an organic-inorganic composite carrier. The commercially available product thereof is exemplified by porous cellulose gel GCL2000, Sephacryl S-1000 prepared by crosslinking allyl dextran and methylene bisacrylamide through a covalent bond, an acrylate carrier Toyopearl, a cross-linked agarose carrier Sepharose CL4B, and a cross-linked cellulose carrier Cellufine. It should be noted, however, that the water-insoluble carrier usable in one or more embodiments of the present invention is not restricted to the carriers exemplified as the above.

It is preferred that the water-insoluble carrier usable in one or more embodiments of the present invention has large surface area and is porous with a large number of fine pores having a suitable size in terms of a purpose and a method of the use of the affinity separation matrix. The carrier may have any form such as beads, monolith, fiber and film including hollow fiber, and any form can be selected.

The above-described ligand is covalently immobilized on a water-insoluble carrier directly or through a linker group. The linker group is exemplified by a C₁₋₆ alkylene group, an amino group (—NH—), an ether group (—O—), a carbonyl group (—C(═O)—), an ester group (—C(═O)—O— or —O—C(═O)—), an amide group (—C(═O)—NH— or —NH—C(═O)—), a urea group (—NHC(═O) NH—); a group formed by binding 2 or more and 10 or less groups selected from the group consisting of a C₁₋₆ alkylene group, an amino group, an ether group, a carbonyl group, an ester group, an amide group and a urea group; and a C₁₋₆ alkylene group having a group selected from the group consisting of an amino group, an ether group, a carbonyl group, an ester group, an amide group and a urea group at one end or both ends. In one or more embodiments, the above-described number of the bound groups is preferably not more than 8 or not more than 6, more preferably 5 or less, and even more preferably 4 or less. The above-described C₁₋₆ alkylene group may be substituted by a substituent such as a hydroxy group.

The affinity separation matrix of one or more embodiments of the present invention can be produced by immobilizing the ligand on a water-insoluble carrier.

With respect to a method for immobilizing the ligand, for example, the ligand can be bound to a carrier by a conventional coupling method utilizing an amino group, a carboxy group or a thiol group of the ligand. Such a coupling method is exemplified by an immobilization method comprising an activation of a carrier by a reaction with cyanogen bromide, epichlorohydrin, diglycidyl ether, tosyl chloride, tresyl chloride, hydrazine, sodium periodate or the like, or introduction of a reactive functional group on the carrier surface, and the coupling reaction between the resulting carrier and a compound to be immobilized as a ligand; and an immobilization method by condensation and crosslinking which method comprises the step of adding a condensation reagent such as carbodiimide or a reagent having a plurality of functional groups in the molecule, such as glutaraldehyde, into a mixture containing a carrier and a compound to be immobilized as a ligand.

A spacer molecule composed of a plurality of atoms may be introduced between the ligand and carrier. Alternatively, the ligand may be directly immobilized on the carrier. Accordingly, the VL-κ-binding peptide of one or more embodiments of the present invention may be chemically modified for immobilization, or may have an additional amino acid residue useful for immobilization. Such an amino acid useful for immobilization is exemplified by an amino acid having a functional group useful for a chemical reaction for immobilization in a side chain, and specifically exemplified by Lys having an amino group in the side chain and Cys having a thiol group in the side chain. In one or more embodiments of the present invention, since the binding ability of the peptide according to one or more embodiments of the present invention to VL-κ is principally maintained in a matrix prepared by immobilizing the peptide as a ligand, any modification and change for immobilization are included in the range of one or more embodiments of the present invention.

It becomes possible by using the affinity separation matrix of one or more embodiments of the present invention that a protein containing a K chain variable region of an immunoglobulin G, i.e. VL-κ-containing protein, is purified in accordance with affinity column chromatography purification method. A VL-κ-containing protein can be purified by a procedure in accordance with a method for purifying an immunoglobulin by affinity column chromatography, for example, such as a method using SpA affinity separation matrix (Non-Patent Document 1).

Specifically, after a solution which contains a VL-κ-containing protein and of which pH is approximately neutral is prepared, the solution is flown through an affinity column packed with the affinity separation matrix of one or more embodiments of the present invention so that the VL-κ-containing protein is selectively adsorbed. Then, an appropriate amount of a pure buffer is flown through the affinity column to wash the inside of the column. At the time, the target VL-κ-containing protein is still adsorbed on the affinity separation matrix of one or more embodiments of the present invention in the column. The affinity separation matrix on which the peptide of one or more embodiments of the present invention is immobilized as a ligand is excellent in the absorption and retention performance of a target VL-κ-containing protein from the step of adding a sample through the step of washing the matrix. Then, an acid buffer of which pH is appropriately adjusted is flown through the column to elute the target VL-κ-containing protein. As a result, purification with high purity can be achieved. Into the acid buffer used for eluting the peptide, a substance for promoting dissociation from the matrix may be added.

The affinity separation matrix of one or more embodiments of the present invention can be reused by allowing an adequate strong acid or strong alkaline pure buffer which do not completely impair the function of the ligand compound or the base material of the carrier to pass through the matrix for washing. In the buffer for reuse, an adequate modifying agent or an organic solvent may be added. Since the affinity separation matrix of one or more embodiments of the present invention is excellent in the chemical stability to an alkaline aqueous solution particularly, it may be preferred that the matrix is washed to be used again by flowing a pure strong alkaline buffer. The frequency of the regeneration procedure by a pure strong alkaline buffer is not necessarily each time after the use, and may be once every five or once every ten.

One or more embodiments of the present invention also relate to a DNA encoding the above-described modified VL-κ-binding peptide. The DNA encoding the peptide of one or more embodiments of the present invention may be any DNA as long as the amino acid sequence produced from translation of the base sequence of the DNA constitutes the peptide. Such a base sequence can be obtained by a common known method, for example, using polymerase chain reaction (hereinafter, abbreviated as “PCR”) technology. Alternatively, such a base sequence can be synthesized by a publicly-known chemical synthesis method or is available from a DNA library. A codon in the base sequence may be substituted by a degenerate codon, and the base sequence is not necessarily the same as the original base sequence as long as the translated amino acids are the same as that encoded by the original base sequence. It is possible to obtain a recombinant DNA having the one or more base sequences, a vector containing the recombinant DNA, such as a plasmid or a phage, a transgenic microorganism or cell transformed by the vector having the DNA, a genetically engineered organisms having the DNA introduced therein, or a cell-free protein synthesis system using the DNA as a template for transcription.

The VL-κ-binding peptide of one or more embodiments of the present invention may be obtained as a fusion peptide fused with a publicly-known protein which beneficially has an action to assist the expression of the protein or to facilitate the purification of the protein. In other words, it is possible to obtain a microorganism or cell containing at least one recombinant DNA encoding a fusion peptide containing the VL-κ-binding peptide of one or more embodiments of the present invention. The above-described protein is exemplified by a maltose-binding protein (MBP) and a glutathione S-transferase (GST), but is not restricted to the exemplified proteins.

Site-specific mutagenesis for modifying the DNA encoding the peptide of one or more embodiments can be conducted by using recombinant DNA technology, PCR method or the like as follows.

For example, a mutagenesis by recombinant DNA technology can be conducted as follows: in the case where there are suitable restriction enzyme recognition sequences on both 16 sides of a target mutagenesis site in the gene encoding the peptide of one or more embodiments, cassette mutagenesis method can be performed. Specifically, a region containing the target mutagenesis site is removed by cleaving the restriction enzyme recognition sites with the restriction enzymes and then a mutated DNA fragment is inserted. Into the mutated DNA fragment, a mutated DNA fragment is introduced only at the target site by a method such as chemical synthesis.

For example, site-directed mutagenesis by PCR can be conducted by double primer mutagenesis. In double primer mutagenesis, PCR is carried out by using a double-stranded plasmid encoding the peptide of one or more embodiments as a template, and using two kinds of synthesized oligo primers which contain complementary mutations in the + strand and − strand.

A DNA encoding a multimer peptide can be produced by ligating the desired number of DNAs each encoding the monomer peptide (single domain) of one or more embodiments of the present invention to one another in tandem. For example, with respect to a method for connecting the DNAs encoding the multimer peptide, a suitable restriction enzyme site is introduced in the DNA sequence and double-stranded DNA fragments cleaved with a restriction enzyme are ligated by using a DNA ligase. One kind of a restriction enzyme site may be introduced or a plurality of restriction enzyme sites of different types may be introduced. When the base sequences encoding each monomer peptide in the DNA encoding the multimer peptide are the same, homologous recombination may be possibly induced in a host. Thus, the sequence identity between base sequences of DNAs encoding the monomer peptides to be connected may be 90% or less, preferably 85% or less, more preferably 80% or less, and even more preferably 75% or less. The sequence identity of a base sequence can be also determined by an ordinary method similarly to an amino acid sequence.

The “expression vector” of one or more embodiments of the present invention contains a base sequence encoding the above-described peptide of one or more embodiments of the present invention or a part of the amino acid sequence of the peptide, and a promoter that can be operably linked to the base sequence to function in a host. Usually, the vector can be constructed by linking or inserting a gene encoding the peptide of one or more embodiments of the present invention to a suitable vector. The vector for insertion of the gene is not particularly restricted as long as the vector is capable of autonomous replication in a host. As such a vector, a plasmid DNA or a phage DNA can be used. For example, in the case of using Escherichia coli as a host, a pQE series vector (manufactured by QIAGEN), a pET series vector (manufactured by Merck), a pGEX series vector (manufactured by GE Healthcare Bioscience) or the like can be used.

The transformant of one or more embodiments of the present invention can be produced by introducing the recombinant vector of one or more embodiments of the present invention into a host cell. A method for introducing the recombinant DNA into a host is exemplified by a method using a calcium ion, electroporation method, spheroplast method, lithium acetate method, agrobacterium infection method, particle gun method and polyethylene-glycol method, but is not restricted thereto. A method for expressing the function of the obtained gene in a host is also exemplified by a method in which the gene according to one or more embodiments of the present invention is implanted into a genome (chromosome). A host cell is not particularly restricted, and bacteria (eubacteria) such as Escherichia coli, Bacillus subtilis, Brevibacillus, Staphylococcus, Streptococcus, Streptomyces and Corynebacterium can be preferably used in terms of mass production in a low cost.

The VL-κ-binding peptide of one or more embodiments of the present invention can be produced by cultivating the above-described transformant in a medium, allowing the transformant to express and accumulate the peptide of one or more embodiments of the present invention in the cultivated bacterial cell or in the culture medium as outside the bacterial cell, and collecting the desired peptide from the culture. Further, the peptide of one or more embodiments of the present invention can also be produced by cultivating the above-described transformant in a medium, allowing the transformant to express and accumulate the fusion protein containing the peptide of one or more embodiments of the present invention in the cultivated bacterial cell or in the culture medium as outside the bacterial cell, collecting the fusion peptide from the culture, cleaving the fusion peptide with a suitable protease, and collecting the desired peptide. A periplasmic space of a bacterial cell is included in the cultivated bacterial cell.

The transformant of one or more embodiments of the present invention can be cultivated in a medium in accordance with a common method for cultivating a host cell. The medium used for cultivating the obtained transformant is not particularly restricted as long as the medium enables high yield production of the peptide of one or more embodiments with high efficiency. Specifically, carbon source and nitrogen source, such as glucose, sucrose, glycerol, polypeptone, meat extract, yeast extract and casamino acid can be used. In addition, an inorganic salt such as potassium salt, sodium salt, phosphate salt, magnesium salt, manganese salt, zinc salt and iron salt is added as required. In the case of an auxotrophic host cell, a nutritional substance necessary for the growth thereof may be added. In addition, an antibiotic such as penicillin, erythromycin, chloramphenicol and neomycin may be added as required.

Furthermore, in order to inhibit the degradation of the target peptide caused by a host-derived protease present inside or outside the bacterial cell, a publicly-known protease inhibitor may be added in an appropriate concentration. The publicly-known protease inhibitor is exemplified by phenylmethane sulfonyl fluoride (PMSF), benzamidine, 4-(2-aminoethyl)-benzenesulfonyl fluoride (AEBSF), antipain, chymostatin, leupeptin, Pepstatin A, phosphoramidon, aprotinin and ethylenediaminetetraacetic acid (EDTA).

In order to obtain rightly folded VL-κ-binding peptide according to one or more embodiments of the present invention, for example, a molecular chaperone such as GroEL/ES, Hsp70/DnaK, Hsp90 and Hsp104/ClpB may be used. For example, such a molecular chaperone is co-existed with the peptide of one or more embodiments by coexpression or as a fusion protein. As a method for obtaining rightly folded present invention peptide, addition of an additive for assisting right folding into the medium and cultivating at a low temperature are exemplified, but the method is not restricted thereto.

The medium for cultivating transformant produced from an Escherichia coli as a host is exemplified by LB medium containing triptone 1%, yeast extract 0.5% and NaCl 1%, 2xYT medium containing triptone 1.6%, yeast extract 1.0% and NaCl 0.5%, or the like.

In one or more embodiments, for example, the transformant may be aerobically cultivated in an aeration-stirring condition at a temperature of 15° C. or higher and 42° C. or lower, preferably 20° C. or higher to 37° C. or lower, for from several hours to several days. As a result, the peptide of one or more embodiments of the present invention is accumulated in the cultivated cell or in the culture liquid as outside the cell to be recovered. In some cases, the cultivation may be performed anaerobically without aeration. In the case where a recombinant peptide is secreted, the produced recombinant peptide can be recovered after the cultivation period by separating the supernatant containing the secreted peptide using a common separation method such as centrifugation and 16 filtration from the cultivated cell. In addition, in the case where the peptide is accumulated in the cultivated cell, the peptide accumulated in the cell can be recovered, for example, by collecting the bacterial cell from the culture liquid by centrifugation, filtration or the like, and then disrupting the bacterial cell by sonication method, French press method or the like, and/or solubilizing the bacterial cell by adding a surfactant or the like. As described above, a periplasmic space of a bacterial cell is included in the cultivated bacterial cell.

A method for purifying the peptide of one or more embodiments can be carried out by any one or an appropriate combination of techniques such as affinity chromatography, cation or anion exchange chromatography, gel filtration chromatography and the like. It can be confirmed whether the obtained purified substance is the target peptide or not by an ordinary method such as SDS polyacrylamide gel electrophoresis, N-terminal amino acid sequence analysis and Western blot analysis.

The present application claims the benefit of the priority date of Japanese patent application No. 2016-92804 filed on May 2, 2016. All of the contents of the Japanese patent application No. 2016-92804 filed on May 2, 2016, are incorporated by reference herein.

### EXAMPLES

Hereinafter, one or more embodiments of the present invention are described in more detail with Examples. The present invention is, however, not restricted to the following Examples in any way, and it is possible to work one or more embodiments of the present invention according to the Examples with an additional appropriate change within the range of the above descriptions and the following descriptions. Such a changed embodiment is also included in the technical scope of the present invention.

The modified peptide obtained in the following Examples is described as “peptide name—introduced mutation”, and wild type into which mutation is not introduced is described as “peptide name—Wild”. For example, pi domain of wild PpL 312 having SEQ ID NO: 7 is described as “LB1-Wild”. In the following Examples, B1 domain of PpL 312 having SEQ ID NO: 12 was mainly used in the experiments. SEQ ID NO: 12 corresponds to an amino acid sequence of SEQ ID NO: 3 with deletion of a N-terminal part and a C-terminal part. SEQ ID NO: 3 is an amino acid sequence of B1 domain of wild PpL 312. SEQ ID NO: 12 is described as “LB1t-Wild” in order to distinguish SEQ ID NO: 12 from SEQ ID NO: 3. Modified B1 domain of PpL 312 of which 41^{st} asparagine is substituted by alanine is described as “LB1t-N41A”. With respect to a mutant having a plurality of mutations, the mutations are described together with a slash. For example, modified B1 domain of PpL 312 into which mutations of N41A and G42A are introduced is described as “LB1t-N41A/G42A”. The number of domain is put down with “d” after a period. For example, a mutant consisting of one domain is described as “LB1t-N41A.1d”.

### Example 1: Preparation of VL-κ-Binding Peptide of Various Modified PpL

(1) Preparation of Expression Plasmid

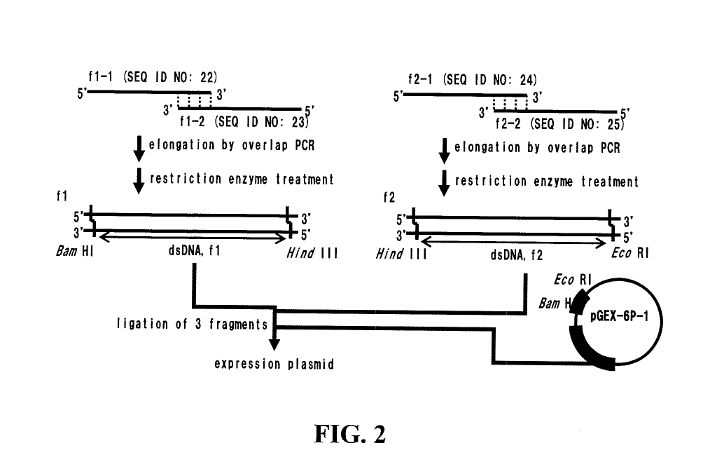

A base sequence of SEQ ID NO: 21 encoding the peptide having the amino acid sequence of LB1t-Wild.1d (SEQ ID NO: 12) was designed by reverse translation from the amino acid sequence. For experimental reasons, the base sequence was designed so that the base sequence encoded an amino acid sequence having Glu-Gln at the N-terminal and Gly at the C-terminal. Such added sequences of 1 to 2 residues can be observed in B1 domain of wild PpL 312. The method for producing the expression plasmid is shown in FIG. 2. A DNA encoding LB1t-Wild.1d was prepared by ligating two kinds of double-stranded DNAs (f1 and f2) having the same restriction enzyme site, and integrated into the multiple cloning site of an expression vector. In fact, the preparation of the peptide-encoding DNA and the integration into the vector were simultaneously performed by three fragments ligation for connecting three double-stranded DNAs of the two kinds of double-stranded DNAs and an expression vector. The two kinds of double-stranded DNAs were prepared by elongating two kinds of single-stranded DNAs (f1-1/f1-2 or f2-1/f2-2) respectively containing about 30-base complementary region with overlapping PCR. Hereinafter, the specific experimental procedure is described. Single-stranded oligo DNAs f1-1 (SEQ ID NO: 22)/f1-2 (SEQ ID NO: 23) were synthesized by outsourcing to Sigma Genosys. The overlapping PCR was performed by using Pyrobest (manufactured by Takara Bio, Inc.) as a polymerase. The PCR product was subjected to agarose electrophoresis and the target band was cut out to extract the double-stranded DNA. The thus extracted double-stranded DNA was cleaved with the restriction enzymes BamHI and HindIII (both available from Takara Bio, Inc.). Similarly, single-stranded oligo DNAs f2-1 (SEQ ID NO: 24)/f2-2 (SEQ ID NO: 25) were synthesized by outsourcing. The double-stranded DNA synthesized by overlapping PCR was extracted and cleaved with the restriction enzymes HindIII and EcoRI (both available from Takara Bio, Inc.). Then, the two kinds of double-stranded DNAs were sub-cloned into the BamHI/EcoRI site in the multiple cloning site of a plasmid vector pGEX-6P-1 (GE Healthcare Bioscience). The ligation reaction for the subcloning was performed by using Ligation high (manufactured by TOYOBO CO., LTD.) in accordance with the protocol attached to the product.

A competent cell (“Escherichia coli HB101” manufactured by Takara Bio, Inc.) was transformed by using the above-described plasmid vector pGEX-6P-1 in accordance with the protocol attached to the competent cell product. By using the plasmid vector pGEX-6P-1, LB1t-Wild.1d which was fused with glutathione-S-transferase (hereinafter, abbreviated as “GST”) could be produced. Then, the plasmid DNA was amplified and extracted by using a plasmid purification kit (“Wizard Plus SV Minipreps DNA Purification System” manufactured by Promega) in accordance with the standard protocol attached to the kit. The base sequence of the peptide-encoding DNA of the expression plasmid was determined by using a DNA sequencer (“3130xl Genetic Analyzer” manufactured by Applied Biosystems). The sequencing PCR was performed by using a gene analysis kit (“BigDye Terminator v. 1.1 Cycle Sequencing Kit” manufactured by Applied Biosystems) and DNA primers for sequencing the plasmid vector pGEX-6P-1 (manufactured by GE Healthcare Bioscience) in accordance with the attached protocol. The sequencing product was purified by using a plasmid purification kit (“BigDye XTerminator Purification Kit” manufactured by Applied Biosystems) in accordance with the attached protocol and used for the base sequence analysis.

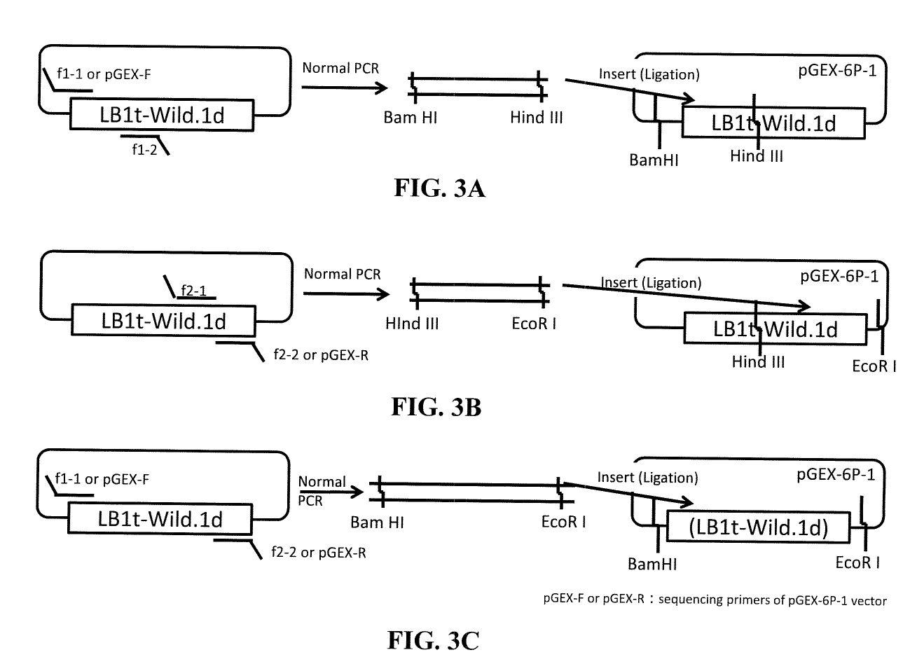

An expression plasmid of various modified LB1t was prepared by PCR using the prepared expression plasmid of LB1t-Wild.1d as a template and a DNA primer for sequencing or any one of single-stranded oligo DNAs of SEQ ID NOs: 22 to 25. The PCR was conducted by using Blend Taq-Plus- (TOYOBO CO., LTD.) in accordance with the enclosed protocol. The double-stranded DNA was cleaved by using two restriction enzymes, and the expression plasmid of LB1t-Wild.1d was also cleaved by using the same restriction enzymes. The cleaved DNA and plasmid were ligated to prepare expression vectors of various modified LB1t. The combination pattern of a DNA primer or a single-stranded oligo DNA and restriction enzymes for preparing a mutant corresponds to any one of patterns of FIG. 3A to 3C. The pattern for preparing various mutants, the base sequences of the used oligo DNA, the base sequences of cDNA encoding mutants, and SEQ ID NOs of amino acid sequences of mutants are shown in Table 1.

TABLE 1

| | | | | Encod- | |
|---|---|---|---|---|---|
| | Preparation | Primer for | Combined | ing | Amino |
| Mutant | method | mutation | primer | DNA | acid |
| LB1t- | FIG. 3B | 26 | pGEX-R | 29 | 32 |
| N41A.1d | | (corresponding | | | |
| | | to f2-1) | | | |
| LB1t- | FIG. 3B | 27 | pGEX-R | 30 | 33 |
| N41K.1d | | (corresponding | | | |
| | | to f2-1) | | | |
| LB1t- | FIG. 3B | 28 | pGEX-R | 31 | 34 |
| G42A.1d | | (corresponding | | | |
| | | to f2-1) | | | |

(2) Production and Purification of Protein

The transformant produced by integrating each of the modified LB1t gene obtained in the above-described (1) was cultivated in 2xYT medium containing ampicillin at 37° C. overnight. The culture solution was inoculated in 2xYT medium containing about 100-fold amount of ampicillin for cultivation at 37° C. for about 2 hours. Then, isopropyl-1-thio-β-D-galactoside, which is hereinafter abbreviated to IPTG, was added so that the final concentration thereof became 0.1 mM, and the transformant was further cultivated at 37° C. for 18 hours.

After the cultivation, the bacterial cell was collected by centrifugation and re-suspended in 5 mL of PBS buffer. The cell was broken by sonication and centrifuged to separate a supernatant fraction as a cell-free extract and an insoluble fraction. When a target gene is integrated into the multiple cloning site of pGEX-6P-1 vector, a fusion peptide having GST added to the N-terminal is produced. Each fraction was analyzed by SDS electrophoresis; as a result, a peptide band assumed to be induced by IPTG was detected at a position 16 corresponding to a molecular weight of about 25,000 or more in the cases of each of all the cell-free extracts obtained from all of the cultured solutions of each transformant.

The GST fusion peptide was roughly purified from each of the cell-free extract containing the GST fusion peptide by affinity chromatography using a GSTrap FF column (GE Healthcare Bioscience), which had an affinity for GST. Specifically, each of the cell-free extract was added to the GSTrap FF column and the column was washed with a standard buffer (20 mM NaH₂PO₄—Na₂HPO₄, 150 mM NaCl, pH 7.4). Then, the target GST fusion peptide was eluted by using an elution buffer (50 mM Tris-HCl, 20 mM Glutathione, pH 8.0).

When a gene is integrated into the multiple cloning site of pGEX-6P-1 vector, an amino acid sequence by which GST can be cleaved by using sequence-specific protease: PreScission Protease (manufactured by GE Healthcare Bioscience) is inserted between GST and a target protein. By using such PreScission Protease, GST was cleaved in accordance with the attached protocol. The target peptide was purified by gel filtration chromatography using a Superdex 75 10/300 GL column (manufactured by GE Healthcare Bioscience) from the GST-cleaved sample used for assay. Each of the reaction mixture was added to the Superdex 75 10/300 GL column equilibrated with a standard buffer, and the target protein therein was separated and purified from the cleaved GST and PreScission Protease. All of the above-described peptide purification by chromatography using the column were performed by using AKTAprime plus system (manufactured by GE Healthcare Bioscience). In addition, after the cleavage of GST, the sequence of Gly-Pro-Leu-Gly-Ser derived from the vector pGEX-6P-1 was added at the N-terminal side of the protein produced in the present example. For example, LB1t-Wild.1d had an amino acid sequence of SEQ ID NO: 12 with Gly-Pro-Leu-Gly-Ser-Glu-Gln at the N-terminal side and Gly at the C-terminal side.

### Comparative Example 1: Preparation and Evaluation of Wild B1 Domain (LB1t-Wild.1d)

Similarly to the method of Example 1, a transformant was prepared by using the expression plasmid of LB1t-Wild.1d prepared in Example 1, and a protein solution was prepared through cultivation and purification.

### Comparative Example 2: Preparation and Evaluation of Modified LB1t as Control

Similarly to the method of Example 1, a transformant was prepared by using the expression plasmid of LB1t-Wild.1d prepared in Example 1, and a protein solution was prepared through cultivation and purification. The combination pattern of a DNA primer or a single-stranded DNA and restriction enzymes corresponds to the pattern of FIG. 3C. The base sequence of the used oligo DNA for preparing various mutants, the base sequences of cDNA encoding mutants, and SEQ ID NOs of amino acid sequences of mutants are shown in Table 2.

TABLE 2

| | Production | Primer for | Combined | Encoding | Amino |
|---|---|---|---|---|---|
| Mutants | method | mutation | primer | DNA | acid |
| LB1t- | FIG. 3C | 35 | pGEX-R | 38 | 41 |
| N06A.1d | | (corresponding | | | |
| | | to f1-1) | | | |
| LB1t- | FIG. 3C | 36 | pGEX-R | 39 | 42 |
| N11A.1d | | (corresponding | | | |
| | | to f1-1) | | | |
| LB1t- | FIG. 3C | 37 | pGEX-R | 40 | 43 |
| G12A.1d | | (corresponding | | | |
| | | to f1-1) | | | |
| LB1t- | FIG. 3C | 44 | pGEX-F | 45 | 46 |
| N56A.1d | | (corresponding | | | |
| | | to f2-2) | | | |

### Example 2: Evaluation of Residual Binding Activity of Various Modified LB1t after Alkaline Treatment on the Basis of Binding Response to aHER-Fab

(1) Alkaline Treatment of Various Modified LB1t

Various dialyzed modified LB1t and wild LB1t were dissolved in water to obtain 20 μM aqueous solution. To 0.04 mL of the aqueous solution, 0.02 mL of 150 mM sodium hydroxide aqueous solution was added so that the final concentration of sodium hydroxide became 50 mM. The mixture was incubated at 25° C. for 12 hours, and then neutralized by using 0.02 mL of 50 mM citric acid (pH 2.4). It is confirmed by using a pH-test paper that the mixture was neutralized.

(2) Preparation of Fab Fragment Derived from IgG

A humanized monoclonal IgG drug product was fragmented into a Fab fragment and a Fc fragment (hereinafter, a Fab 16 fragment and a Fc fragment are respectively abbreviated as “Fab” and “Fc”) by using papain, and only Fab was purified. Specifically, anti-HER2 monoclonal IgG of which light chain was κ chain (general name: “trastuzumab”) was dissolved in a buffer for papain digestion (0.1 M AcOH—AcONa, 2 mM EDTA, 1 mM cysteine, pH 5.5). Agarose on which papain was immobilized (“Papain Agarose from papaya latex” manufactured by SIGMA) was added thereto. The mixture was incubated at 37° C. for about 8 hours while the mixture was stirred by a rotator. The reaction mixture containing both of Fab fragment and Fc fragment was separated from the agarose on which papain was immobilized. The aHER-Fab was obtained as a flow-through fraction by affinity chromatography using MabSelect SuRe column manufactured by GE Healthcare Bioscience from the reaction mixture. The obtained aHER-Fab solution was subjected to purification by gel filtration chromatography using Superdex 75 10/300 GL column to purify aHER-Fab. A standard buffer was used for equilibrating and separation. The above protein purification by chromatography was performed by using AKTAavant 25 system as Example 1.

(3) Measurement of Binding Response of Various Modified LB1t to aHER-Fab

The binding response of various modified LB1t obtained in the above-described Example 1(2) to aHER-Fab was evaluated by using a biosensor Biacore 3000 (manufactured by GE Healthcare Bioscience) utilizing surface plasmon resonance. In the present Example, the aHER-Fab obtained in the above-described Example 2(2) was immobilized on a sensor tip, and each of the peptide was flown on the tip to detect the interaction between 16 the two. The aHER-Fab was immobilized on a sensor tip CM5 by amine coupling method using N-hydroxysuccinimide (NHS) and N-ethyl-N′-(3-dimethylaminopropyl)carbodiimide hydrochloride (EDC), and ethanolamine was used for blocking. All of the sensor tip and reagents for immobilization was manufactured by GE Healthcare Bioscience. The aHER-Fab solution was diluted to about 10 times by using a buffer for immobilization (10 mM CK₃COOH—CH₃CONa, pH 4.5), and the aHER-Fab was immobilized on the sensor tip in accordance with the protocol attached to the Biacore 3000. In addition, a reference cell as negative control was also prepared by activating another flow cell on the tip with EDC/NHS and then immobilizing ethanolamine only. An amount of immobilized aHER-Fab was about 5000 RU. By adjusting an immobilized amount to high as 5000 RU or more and a flow rate to be slow, a detection sensitivity and a dependence of an analyte concentration are improved. In other words, under the environment where mass transport limitation is applied, the dependence of a binding response on an affinity is decreased and the dependence on a concentration is relatively increased.

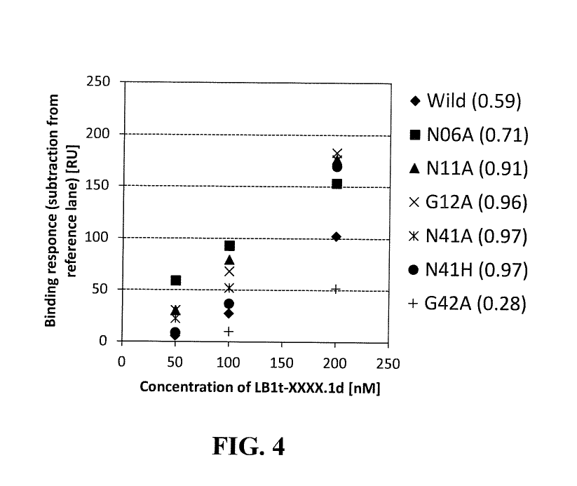

Various modified LB1t solutions of 50 nM, 100 nM or 200 nM before an alkaline treatment were prepared by using a running buffer (20 mM NaH₂PO₄—Na₂HPO₄, 150 mM NaCl, 0.005% P-20, pH 7.4). The solution was added to the sensor tip in a flow rate of 10 μL/min for 2 minutes. Binding response curves at the time of addition (association phase, for 2 minutes) and after the addition (dissociation phase, for 2 minutes) were sequentially obtained at a measurement temperature of 25° C. After each measurement, about 20 mM NaOH was added for 16 washing. A binding response 1 minute after the addition (resonance unit value of a binding response curve) was plotted on a vertical axis of a graph, and the added analyte concentration at the addition was plotted on an abscissa axis. The graph is shown as FIG. 4. Thus, in the concentration range of the evaluation system, a bonding response is somewhat proportional to an analyte concentration. The slope value of various modified LB1t in the graph is shown in parentheses of FIG. 4. For example, when the concentration of LB1t-N41A.1d is increased by 1 nM, the binding response 1 minute after the addition is increased by about 1 RU. As the graphs, the increase degree of the binding response to the analyte concentration is dependent on the mutant. In this evaluation system, a mere ratio of responses before and after the alkaline treatment was not used, but after correcting to convert to concentration, a residual binding activity was calculated.

(4) Residual Binding Activity of Various Modified LB1t after Alkaline Treatment to aHER-Fab

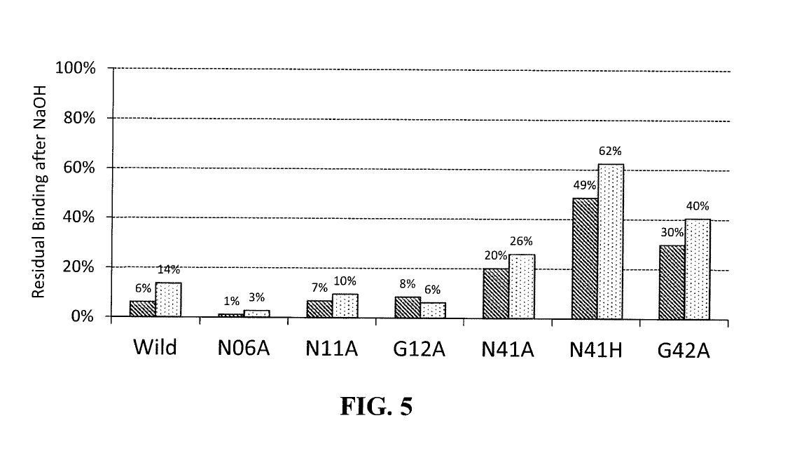

A concentration of various modified LB1t after the alkaline treatment was adjusted to 200 nM by using the running buffer, and the solution was added to the sensor tip in a flow rate of 10 μL/min for 2 minutes similarly to the above to measure the binding response to aHER-Fab 1 minute after the addition. An analyte concentration was calculated from the binding response value after the alkaline treatment and the slope determined on the basis of the measured binding response value at 200 nM before the alkaline treatment and the graph of 16 FIG. 4. In addition, a concentration ratio of the analyte concentration as a mutant concentration of which binding activity was maintained to the concentration before the alkaline treatment as 100% was calculated as a residual binding activity. With respect to each modified LB1t, the graph of residual binding activity value of prepared two alkaline-treated samples is demonstrated as FIG. 5.

Though variations in residual binding activity value between samples is recognized, it is clear that the residual binding activities of LB1t-N41A.1d, LB1t-N41H.1d and LB1t-G42A.1d after the alkaline treatment are significantly higher in comparison with that of LB1t-Wild.1d (Comparative example 1). As described above, it is known that the resistance of Protein A (SpA) to alkali is increased by substituting an asparagine residue (Asn) and a glycine residue (Gly) after Asn. On the one hand, as the residual binding activities of LB1t-NO6A.1d, LB1t-N11A.1d and LB1t-G12A.1d after the alkaline treatment (Comparative example 2), the resistance to alkali is amazingly decreased by the mutation of Asn and Gly after Asn. It was a particularly excellent aspect of one or more embodiments of the present invention that a mutation part to improve a resistance to alkali is specified by the result.

### Example 3: Evaluation of Residual Binding Activity of Various Modified LB1t after Alkaline Treatment on the Basis of Binding Response to aIgE-Fab

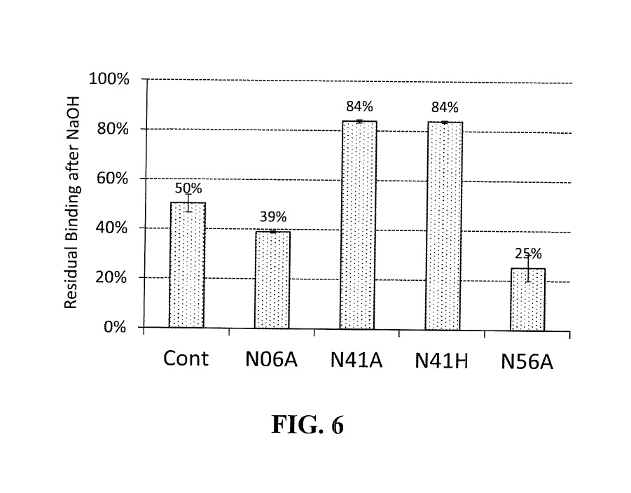

Experiment was conducted similarly to the above-described Example 2 except that anti-IgE monoclonal IgG (general name: “omalizumab”) was used in place of anti-HER2 monoclonal IgG (general name: “trastuzumab”). The result is shown in FIG. 6.

As the result shown by FIG. 6, it was also proved that the residual binding activities of LB1t-N41A.1d and LB1t-N41H.1d are significantly higher than that of LB1t-Wild.1d (Comparative example 1) with respect to other kind of Fab. In addition, it was demonstrated again that even when Asn at the position except for the 41^{st} position is substituted, such an effect is not tended to be exhibited.

Although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. An immunoglobulin κ chain variable region-binding peptide selected from the group consisting of:
a first immunoglobulin κ chain variable region-binding peptide comprising an amino acid sequence of SEQ ID NO: 20 with at least one substitution at one or more positions selected from the group consisting of the 41^{st} position and the 42^{nd} position;
a second immunoglobulin κ chain variable region-binding peptide comprising the amino acid sequence of the first peptide, the amino acid sequence further comprising 1 to 20 amino acid deletions, substitutions and/or additions at one or 16 more positions except for the 41^{st} position and the 42^{nd} position, wherein the second peptide has a higher chemical stability to an alkaline aqueous solution than a chemical stability of the first peptide before introducing the at least one substitution; and
a third immunoglobulin κ chain variable region-binding peptide comprising an amino acid sequence having a sequence identity of 80% or more with the amino acid sequence of the first peptide, provided that the at least one substitution is not further mutated, wherein the third peptide has a higher chemical stability to an alkaline aqueous solution than the chemical stability of the first peptide before introducing the at least one substitution.

2. The immunoglobulin κ chain variable region-binding peptide according to claim 1, wherein the amino acid sequence of the first peptide is selected from the group consisting of amino acid sequences of SEQ ID NOs: 12 to 19.

3. The immunoglobulin κ chain variable region-binding peptide according to claim 1, wherein the amino acid residue at the 41^{st} position is substituted in the amino acid sequence of the first peptide.

4. The immunoglobulin κ chain variable region-binding peptide according to claim 1, wherein the amino acid residue at the 41^{st} position is substituted by A1a or His and the amino acid residue at the 42^{nd} position is substituted by Ala, in the amino acid sequence of the first peptide.

5. The immunoglobulin κ chain variable region-binding peptide according to claim 1,
wherein the immunoglobulin κ chain variable region-binding peptide is the second peptide, and
wherein the 1 to 20 amino acid deletions, substitutions and/or additions are located at N-terminal and/or C-terminal.

6. The immunoglobulin κ chain variable region-binding peptide according to claim 1,
wherein the immunoglobulin κ chain variable region-binding peptide is the third peptide, and
wherein the sequence identity is 95% or more.

7. An immunoglobulin κ chain variable region-binding peptide multimer, comprising the two or more immunoglobulin κ chain variable region-binding peptides according to claim 1 as domains, wherein the immunoglobulin κ chain variable region-binding peptides are connected one another.

8. An affinity separation matrix, wherein the immunoglobulin κ chain variable region-binding peptide according to claim 1 is immobilized as a ligand on a water-insoluble carrier.

9. A method for producing a protein comprising a immunoglobulin κ chain variable region, comprising:
contacting a liquid sample comprising a protein comprising an immunoglobulin κ chain variable region with the affinity separation matrix according to claim 8, adsorbing the protein on the affinity separation matrix; and
separating the protein adsorbed on the affinity separation matrix from the affinity separation matrix.

10. A DNA, encoding the immunoglobulin κ chain variable region-binding peptide according to claim 1.

11. A vector, comprising the DNA according to claim 10.

12. A transformant, transformed by the vector according to claim 11.
